(19) United States
(12) Patent Application Publication
(10) Pub. No.: US 2025/0359606 A1
(43) Pub. Date: Nov. 27, 2025

(54) Hands-Free Pumping Bra and Method

(71) Applicant: Motif Medical, LLC, Asheville, NC (US)

(72) Inventors: Eric Robert Peterson, Raleigh, NC (US); Kara Ashley Nemeth, Weaverville, NC (US)

(73) Assignee: Motif Medical, LLC, Asheville, NC (US)

(21) Appl. No.: 19/214,889

(22) Filed: May 21, 2025

Related U.S. Application Data
(60) Provisional application No. 63/651,031, filed on May 23, 2024.

(51) Int. Cl. A41C 3/04; A44B 13/00; A44B 19/26; A61M 1/06

(52) U.S. Cl. CPC A41C 3/04; A44B 13/00; A44B 19/26; A61M 1/067

(57) ABSTRACT

A hands-free pumping bra. The hands-free pumping bra may include a first body panel configured to be disposed over a first breast of a wearer, the first body panel may include a first opening; and a first covering coupled to the first body panel to cover the first opening, the first covering may include a first flap attached to the first body panel and covering a first portion of the first opening, a second flap attached to the first body panel and covering a second portion of the first opening, and a third flap attached to the first body panel and covering a third portion of the first opening, the first flap, the second flap, and the third flap at least partially overlap and collectively are required to cover the entire first opening, the first covering providing a first access opening to receive therethrough a portion of a breast pump.

### RELATED APPLICATIONS

This application is related and claims priority to U.S. Provisional Patent Application No.: 63/651,031, filed on May 23, 2024, the application of which is incorporate herein by reference in its entirety.

### TECHNICAL FIELD

The subject matter of the invention relates generally to devices and methods for pumping breast milk and more particularly to a hands-free pumping bra and methods.

### BACKGROUND

Nursing mothers often use breast pumps to express milk to feed their infant. When using a breast pump, a nursing mother must manually hold the breast flange or shield over the exposed portion of the breast. Further, when using a breast pump, a woman will often express milk from both breasts simultaneously to minimize the length of time it takes to express milk. To express milk from both breasts, the woman must hold the breast flange or shield with each hand against her breast. This leaves the nursing mother in an awkward position without the full use of both hands. Making it difficult to do simple tasks such as operating the breast pump. As a result, new approaches are needed with respect to hands-free operation of breast pumps.

### SUMMARY

In one embodiment, a hands-free pumping bra is provided. The hands-free pumping bra may include a first body panel configured to be disposed over at least a portion of a first breast of a wearer, the first body panel may include a first opening; and a first covering coupled to the first body panel and disposed to cover the first opening, the first covering may include a first flap attached to the first body panel and covering a first portion of the first opening, a second flap attached to the first body panel and covering a second portion of the first opening, and a third flap attached to the first body panel and covering a third portion of the first opening, wherein the first flap, the second flap, and the third flap at least partially overlap one another and collectively are required to cover the entire first opening, the first covering providing a first access opening configured to receive therethrough a portion of a first breast pump. The first opening may be substantially circular. The first flap, the second flap, and the third flap may be configured such that, when received therethrough, the received portion of the first breast pump is held in place without hand-held support. The first opening may be defined by an arrangement of reinforced stitching. The first flap, the second flap, and the third flap each may include a generally semicircular shape. The \ first flap, the second flap, and the third flap may be layered and orientated one to another such that all three together are required to cover the entire circular area of the first opening, wherein an absence of any one of the first flap, the second flap, or the third flap would leave a portion of the first opening uncovered. The grain of a fabric of each of the first flap, the second flap, and the third flap may be orientated to be substantially parallel to a straight edge of each of its respective one of the first flap, the second flap, and the third flap. The garment may further include a second body panel configured to be disposed over at least a portion of a second breast of a wearer, the second body panel may include a second opening; and a second covering coupled to the second body panel and disposed to cover the second opening, the second covering may include a fourth flap attached to the second body panel and covering a first portion of the second opening, a fifth flap attached to the second body panel and covering a second portion of the second opening, and a sixth flap attached to the second body panel and covering a third portion of the second opening, wherein the fourth flap, the fifth flap, and the sixth flap at least partially overlap one another and collectively are required to cover the entire second opening, the second covering providing a second access opening configured to receive therethrough a portion of the first breast pump or a portion of a second breast pump. The second opening may be substantially circular. The fourth flap, the fifth flap, and the sixth flap may be configured such that when received therethrough the received portion of the first breast pump or the second breast pump may be held in place without hand-held support. The second opening may be defined by an arrangement of reinforced stitching. The fourth flap, the fifth flap, and the sixth flap each comprise a generally semicircular shape. The fourth flap, the fifth flap, and the sixth flap may be layered and orientated one to another such that all three together are required to cover the entire circular area of the second opening, wherein an absence of any one of the fourth flap, the fifth flap, or the sixth flap would leave a portion of the second opening uncovered. The grain of a fabric of each of the fourth flap, the fifth flap, and the sixth flap may be orientated to be substantially parallel to a straight edge of each of its respective one of the fourth flap, the fifth flap, and the sixth flap. The garment may further include a front fastener, wherein the front fastener is configured to fasten the first body panel and second body panel together at a front portion of the garment. The front fastener may include a zipper. The garment may further include a rear fastener, wherein the rear fastener may be configured to fasten the first body panel and second body panel together in a back portion of the garment. The rear fastener may include one of or a combination of a hook and eye style fastener or a hook and loop style fastener.

In another embodiment, a method of using a hands-free pumping bra is provided. The method may include providing a hands-free pumping bra garment, The hands-free pumping bra may include a first body panel configured to be disposed over at least a portion of a first breast of a wearer, the first body panel including a first opening; and a first covering coupled to the first body panel and disposed to cover the first opening, the first covering may include a first flap attached to the first body panel and covering a first portion of the first opening, a second flap attached to the first body panel and covering a second portion of the first opening, and a third flap attached to the first body panel and covering a third portion of the first opening, wherein the first flap, the second flap, and the third flap at least partially overlap and collectively are required to cover the entire first opening, the first covering providing a first access opening configured to receive therethrough a portion of a first breast pump. The method may further include putting on the hands-free pumping bra garment by a user; inserting a portion of the first breast pump into the first opening through the first flap, the second flap, and the third flap; and supporting the inserted portion of the first breast pump in a handsfree manner via the first flap, second flap, and third flap. The hands-free pumping bra garment may further include a second body panel configured to be disposed over at least a portion of a second breast of a wearer, the second body panel may include a second opening; and a second covering coupled to the second body panel and disposed to cover the second opening, the second covering may include a fourth flap attached to the second body panel and covering a first portion of the second opening, a fifth flap attached to the second body panel and covering a second portion of the second opening, and a sixth flap attached to the second body panel and covering a third portion of the second opening, wherein the fourth flap, the fifth flap, and the sixth flap at least partially overlap and collectively are required to cover the entire second opening, the second covering providing a second access opening configured to receive therethrough a portion of the first breast pump or a portion of a second breast pump. The method may further include inserting a portion of the first breast pump or the second breast pump into the second opening through the fourth flap, the fifth flap, and the sixth flap; and holding the inserted portion of the first breast pump or the second breast pump in a handsfree manner via the fourth flap, the fifth flap, and the sixth flap.

In yet another embodiment, a hands-free pumping bra is provided. The hands-free pumping bra may include a body portion configured to be disposed over at least a portion of a breast of a wearer, the body portion may include an opening, wherein the opening is configured to be in substantial alignment with a nipple region of a wearer; and a covering coupled to the body portion and disposed to cover the opening, the covering including three overlapping substantially semicircular-shaped flaps attached to the body portion, wherein the three overlapping semicircular-shaped flaps collectively are required to cover the entire opening, the covering providing an access opening configured to receive therethrough a portion of a breast pump.

### BRIEF DESCRIPTION OF THE DRAWINGS

Having thus described the subject matter of the invention in general terms, reference will now be made to the accompanying drawings, which are not necessarily drawn to scale, and wherein:

FIG. 1 illustrates an example of a hands-free pumping bra in use with a breast pump, in accordance with an embodiment of the invention;

FIG. 2, FIG. 3, and FIG. 4 illustrate a front perspective view, a front plan view, and a back plan view, respectively, of an example of a hands-free pumping bra in a fastened state, in accordance with an embodiment of the invention;

FIG. 5, FIG. 6, and FIG. 7 illustrate a front perspective view, a front plan view, and a back plan view, respectively, of the hands-free pumping bra shown in FIG. 2, FIG. 3, and FIG. 4 in an unfastened state, in accordance with an embodiment of the invention;

FIG. 8, FIG. 9, and FIG. 10 illustrate a front perspective view, a front plan view and cross-sectional view, and a back plan view, respectively, of an example of a front opening of the hands-free pumping bra including three overlapping flaps, in accordance with an embodiment of the invention;

FIG. 11A, FIG. 11B, and FIG. 11C illustrate front views showing the three individual flaps, respectively, of the front opening of the hands-free pumping bra, in accordance with an embodiment of the invention;

FIG. 12, FIG. 13, and FIG. 14 illustrate another front perspective view, front plan view, and back plan view, respectively, of the front opening of the hands-free pumping bra including the three overlapping flaps and indicating example grain patterns thereof, in accordance with an embodiment of the invention;

FIG. 15A through FIG. 15H illustrate front plan views showing examples of different sizes of the hands-free pumping bra, in accordance with embodiments of the invention;

FIG. 16, FIG. 17, and FIG. 18 illustrate a front perspective view, a front plan view, and a back plan view, respectively, of another example of a hands-free pumping bra in a fastened state, in accordance with an embodiment of the invention;

FIG. 19, FIG. 20, and FIG. 21 illustrate a front perspective view, a front plan view, and a back plan view, respectively, of the hands-free pumping bra shown in FIG. 16, FIG. 17, and FIG. 18 in an unfastened, in accordance with an embodiment of the invention;

FIG. 22, FIG. 23, and FIG. 24 illustrate a front perspective view, a front plan view, and a back plan view, respectively, of another example of a hands-free pumping bra in a fastened state, in accordance with an embodiment of the invention;

FIG. 25, FIG. 26, and FIG. 27 illustrate a front perspective view, a front plan view, and a back plan view, respectively, of the hands-free pumping bra shown in FIG. 22, FIG. 23, and FIG. 24 in an unfastened state, in accordance with an embodiment of the invention; and

FIG. 28 illustrates a flow diagram of an example of a method of using the hands-free pumping bra, in accordance with an embodiment of the invention.

### DETAILED DESCRIPTION

The subject matter of the invention now will be described more fully hereinafter with reference to the accompanying drawings, in which some, but not all embodiments of the subject matter of the invention are shown. Like numbers refer to like elements throughout. The subject matter of the invention may be embodied in many different forms and should not be construed as limited to the embodiments set forth herein; rather, these embodiments are provided so that this disclosure will satisfy applicable legal requirements. Indeed, many modifications and other embodiments of the subject matter of the invention set forth herein will come to mind to one skilled in the art to which the subject matter of the invention pertains having the benefit of the teachings presented in the foregoing descriptions and the associated drawings. Therefore, it is to be understood that the subject matter of the invention is not to be limited to the specific embodiments disclosed and that modifications and other embodiments are intended to be included within the scope of the appended claims.

In some embodiments, the subject matter of the invention provides a hands-free pumping bra and methods.

In some embodiments, the hands-free pumping bra and methods provide a bra body including two substantially circular front openings designed to receive and hold milk collection bottles.

In some embodiments, the hands-free pumping bra and methods provide a bra body including two substantially circular front openings and wherein each front opening may include three overlapping semicircular-shaped front opening flaps.

In some embodiments, the hands-free pumping bra and methods provide a bra body including two substantially circular front openings and wherein each front opening may include three overlapping semicircular-shaped front opening flaps and wherein the three overlapping flaps are designed for holding a portion of a breast pump, for example, a milk collection bottle, in hands-free fashion.

In some embodiments, the hands-free pumping bra and methods provide a bra body including two substantially circular front openings and wherein each front opening may include three overlapping semicircular-shaped front opening flaps and wherein the three overlapping flaps are sized and positioned one to another such that all three together are required to span the entire circular area of the front opening; that is if any one flap were missing then some portion of the front opening would be exposed or open.

Further, a method is provided of using the hands-free pumping bra including two front openings and each front opening including three overlapping flaps.

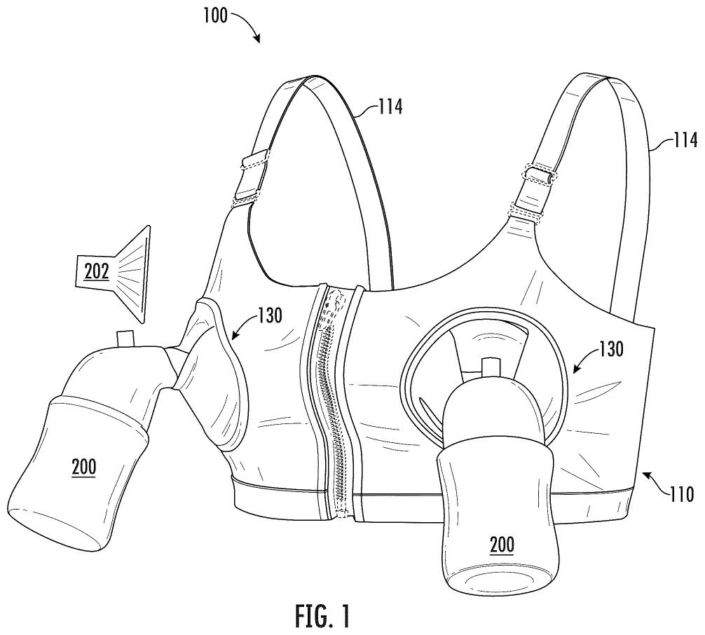

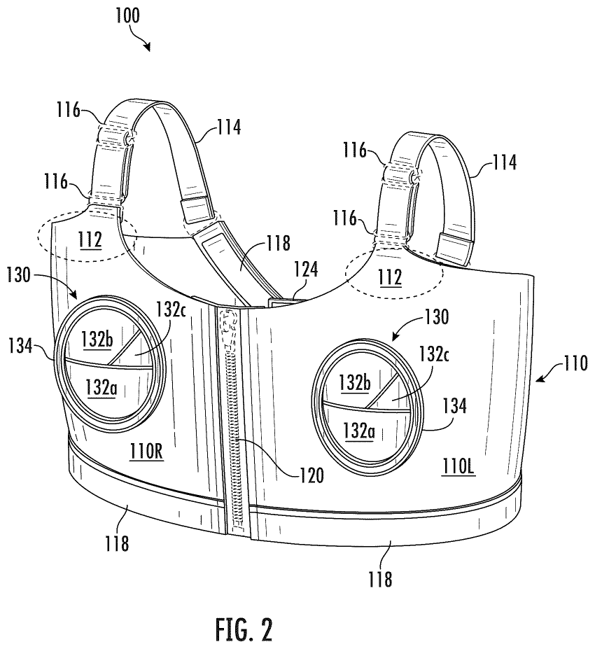

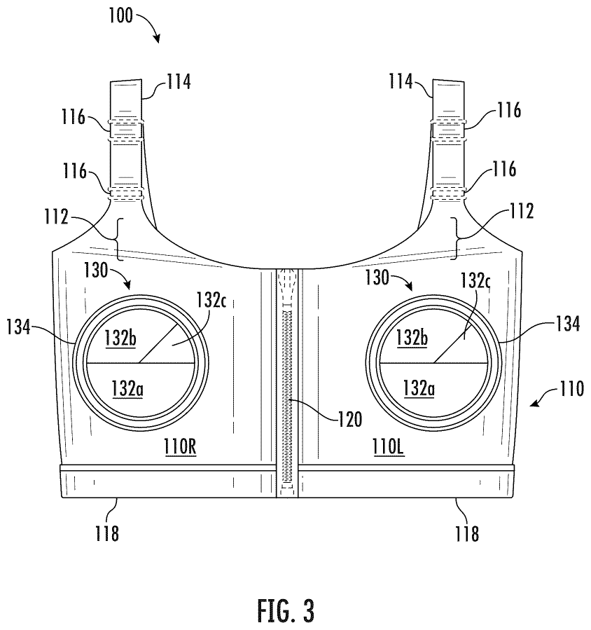

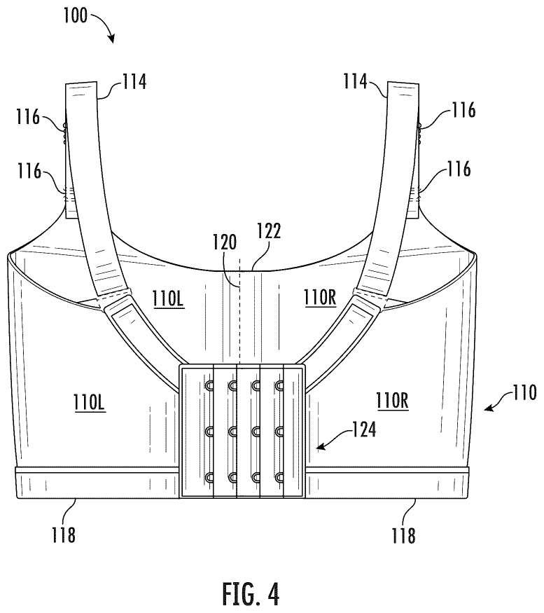

Referring now to FIG. 1 is an example of a hands-free pumping bra 100 in use with a portion of a breast pump, e.g., milk collection bottles, in accordance with an embodiment of the invention. In this example, hands-free pumping bra 100 may generally include a two-piece bra body 110 with bra straps 114. Further, bra body 110 may include two substantially circular front openings 130. The two front openings 130 are breast pump openings designed to receive milk collection bottles 200 that may be fluidly connected to a breast pump (not shown). For example, each milk collection bottle 200 may include a flange 202 that may be fitted into and through a front opening 130 and against a woman's breast. More details of examples of hands-free pumping bra 100 are shown and described below in FIG. 2 through FIG. 27.

Further, in hands-free pumping bra 100, each of the front openings 130 may include an arrangement of three overlapping flaps, which are three overlapping pieces of flexible material, through which flange 202 may engage for holding milk collection bottle 200. The arrangement of the three overlapping flaps provides a suitably secure and strong mechanism for holding a milk collection bottle 200 without hand-held support (i.e., held in hands-free fashion). For example, flange 202 may be pushed through a center or intersecting portion of the three overlapping flaps and held.

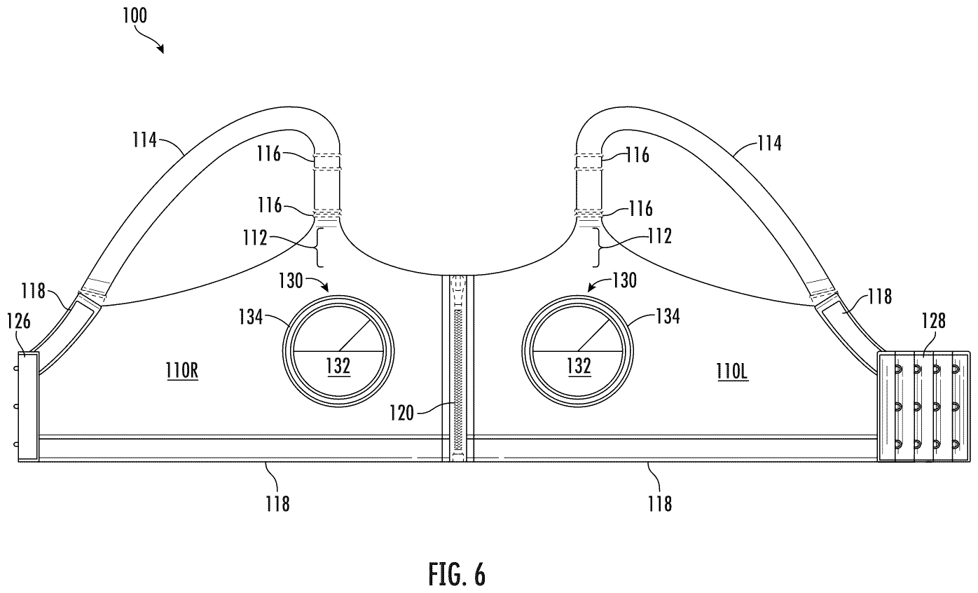

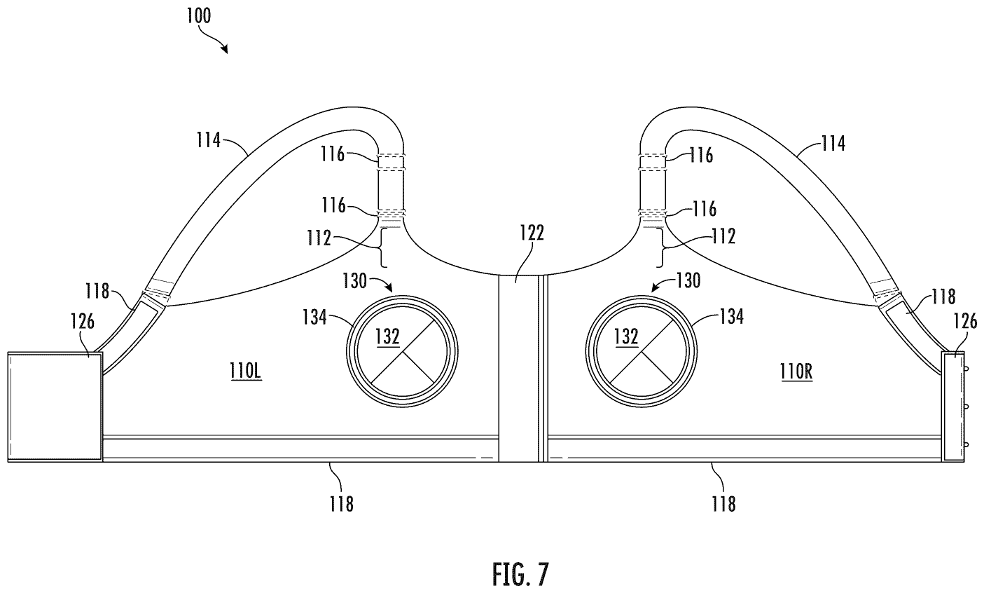

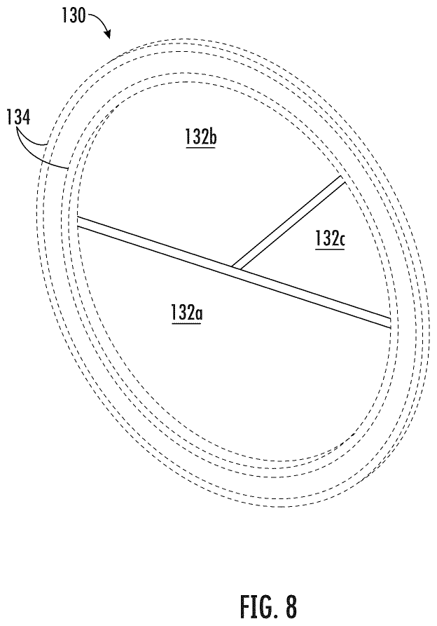

In one embodiment the three overlapping flaps of each of the front openings 130 of hands-free pumping bra 100 may be sized and positioned one to another such that all three together are required to span the entire circular area of front opening 130. Put another way, if any one flap were missing, then some portion of front opening 130 would be exposed or open. Any two flaps alone will not span the entire circular area of front opening 130. More details of an example of front openings 130 are shown and described below in FIG. 8 through FIG. 14.

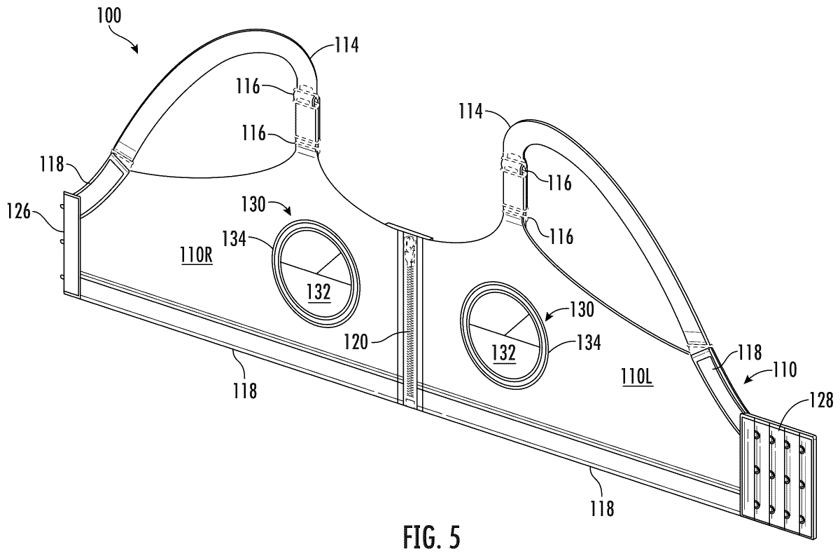

Referring now to FIG. 2 through FIG. 7 is various views of one example of a hands-free pumping bra 100, in accordance with an embodiment of the invention. In this example, the hands-free pumping bra 100 may include a hook and eye system for fastening in the back. For example, FIG. 2, FIG. 3, and FIG. 4 show a perspective view, a front view, and a back view, respectively, of the hands-free pumping bra 100 in a fastened state via the hook and eye system. Further, FIG. 5, FIG. 6, and FIG. 7 show a perspective view, a front plan view, and a back plan view, respectively, of the hands-free pumping bra shown in FIG. 2, FIG. 3, and FIG. 4 in an unfastened from the back state.

In this example, hands-free pumping bra 100 may include two-piece bra body 110 formed, for example, by a right body panel 110R and a left body panel 110L. Further, an upper portion of each of the right body panel 110R and left body panel 110L may include a body transition portion 112. Each of the two body transition portions 112 may include a tapered shape for transitioning to a bra strap 114. Further, each bra strap 114 may include one or more buckles 116 for adjusting the bra strap 114. Further, certain upper and lower portions of right body panel 110R and left body panel 110L may include some amount of edging (or border) fabric 118.

Right body panel 110R, left body panel 110L, and/or edging (or border) fabric 118 may be formed of any materials and/or fabrics commonly used for making bras. These materials may include, but are not limited to, cotton, silk, wool, duoplex fabric (i.e., a low-stretch knit polyester material), elastane (i.e., a polymer fiber), spandex material, nylon, modal material, stretch mesh (i.e., a blend of nylon and spandex), plush material, and/or the like. Further, bra straps 114 may be made of, for example, nylon, spandex, and/or other suitable materials.

Further, right body panel 110R and left body panel 110L may be fastened together in the front via a front zipper 120 (or other suitable mechanism and/or technique). A zipper flap 122 may be provided on the inside (or body side) of front zipper 120. Additionally, right body panel 110R and left body panel 110L may be fastened together in the back via a hook and eye system 124. In this way, together right body panel 110R and left body panel 110L may form bra body 110. In one example, hook and eye system 124 may include an arrangement of hook fasteners 126 on one end of right body panel 110R and an arrangement of eye fasteners 128 on one end of left body panel 110L, as shown in FIG. 5, FIG. 6, and FIG. 7, or vice versa. In one example, a line of three hook fasteners 126 may be provided and a 3×4 array of eye fasteners 128 may be provided. The 3×4 array of eye fasteners 128 provides four choices of adjustment for providing the best fit. Other array arrangements of hook fasteners 126 and eye fasteners 128 may be provided and may include an array of greater or less than a 3×4.

Further, while front zipper 120 may be provided for ease of taking hands-free pumping bra 100 on and off, front zipper 120 may be optional. That is, in other embodiments, hands-free pumping bra 100 may include hook and eye system 124 in the back only. A zipper flap 122 may also (or alternatively) be provided on the outside of front zipper 120.

Again, hands-free pumping bra 100 may include two front openings 130 (or breast pump openings). For example, one front opening 130 in the front of right body panel 110R and another front opening 130 in the front of left body panel 110L. Each of the front openings 130 may include an arrangement of three overlapping front opening flaps 132, which are three overlapping pieces of, for example, flexible material. In one example, each of the front openings 130 may include front opening flaps 132a, 132b, and 132c. Each of the front openings 130 may be defined or bounded by reinforced stitching 134.

The arrangement of the three overlapping front opening flaps 132a, 132b, and 132c provides a suitably secure and strong mechanism for holding a milk collection bottle 200 without hand-held support (i.e., held in hands-free fashion). For example, flange 202 may be pushed through a center or intersecting portion of the three overlapping front opening flaps 132a, 132b, and 132c and held.

Referring now to FIG. 8 through FIG. 14 is various views showing more details of an example of front openings 130. For example, FIG. 8, FIG. 9, and FIG. 10 show a perspective view, a front view, and a back view, respectively, of an example of front opening 130 including an arrangement of the three overlapping front opening flaps 132a, 132b, and 132c. Again, each front opening 130 may be defined by reinforced stitching 134.

Each of the front opening flaps 132a, 132b, and 132c within front opening 130 may be a semicircular (or other suitable shape) piece of material. In one embodiment, front opening flaps 132 may be arranged in order from front to back with front opening flap 132a being frontmost, then front opening flap 132b, and then front opening flap 132c being backmost, as shown in FIG. 8, FIG. 9, and FIG. 10. In one example, the material forming front opening flaps 132 may be the same material used to form right body panel 110R and left body panel 110L.

Further, FIG. 9 shows a cross-sectional view of front opening 130 taken along a line AA and a cross-sectional view of front opening 130 taken along a line BB. Both indicating the front to back order of front opening flap 132a, then front opening flap 132b, and then front opening flap 132c. Further, FIG. 9 shows that the circular inner area of front opening 130 bounded by reinforced stitching 134 has diameter d and a radius r. Diameter d may be in the range from about 6 cm to about 8 cm in one example, or may be about 7 cm in another example. Accordingly, radius r may be in the range from about 3 cm to about 4 cm in one example, or may be about 3.5 cm in another example. Front opening 130 with a diameter d of about 7 cm may be suitably small for supporting and holding the flange of a milk collection bottle (see FIG. 1) while pumping. At the same time a 7 cm-front opening 130 is suitably large to allow the flange to be inserted easily.

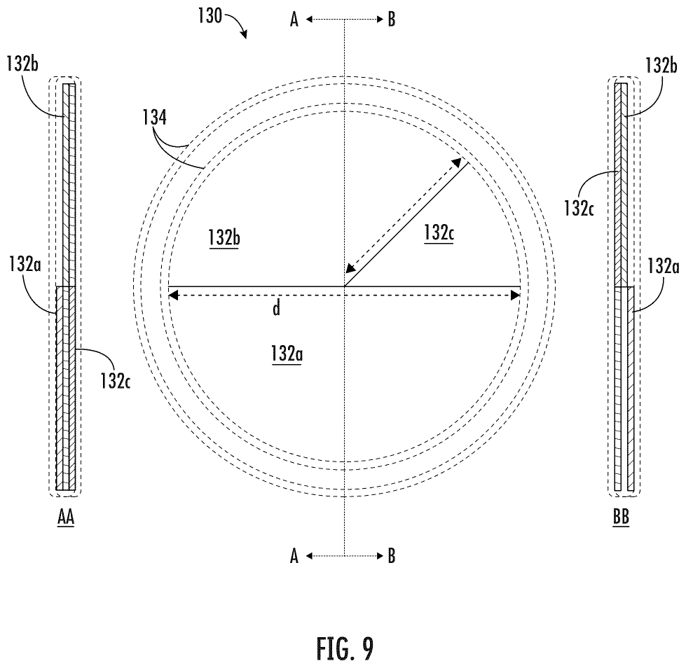

While each of the front opening flaps 132a, 132b, and 132c may have substantially the same semicircular shape, the orientation of one to another is shown in FIG. 11A, FIG. 11B, and FIG. 11C. For example, and referring now to FIG. 11A, the straight edge of front opening flap 132a may be considered a reference plane p set at 0 degrees. Next, and referring now to FIG. 11B, the straight edge of front opening flap 132b may be set at about 135 degrees clockwise (cw) with respect to the reference plane p of front opening flap 132a. Next, and referring now to FIG. 11C, the straight edge of front opening flap 132c may be set at about 225 degrees clockwise (cw) with respect to the reference plane p of front opening flap 132a.

Referring still to FIG. 11A, FIG. 11B, and FIG. 11C, in one embodiment, the three overlapping front opening flaps 132 are sized and positioned one to another such that all three together are required to span the entire circular area of front opening 130. Put another way, if any one front opening flap 132a, 132b, or 132c were missing, then some portion of front opening 130 would be exposed or open. This is because any two front opening flaps 132 alone will not span the entire circular area of front opening 130.

For example, front opening flaps 132a and 132b alone without front opening flap 132c will leave some portion of front opening 130 exposed or open. Likewise, front opening flaps 132a and 132c alone without front opening flap 132b will leave some portion of front opening 130 exposed or open. Likewise, front opening flaps 132b and 132c alone without front opening flap 132a will leave some portion of front opening 130 exposed or open.

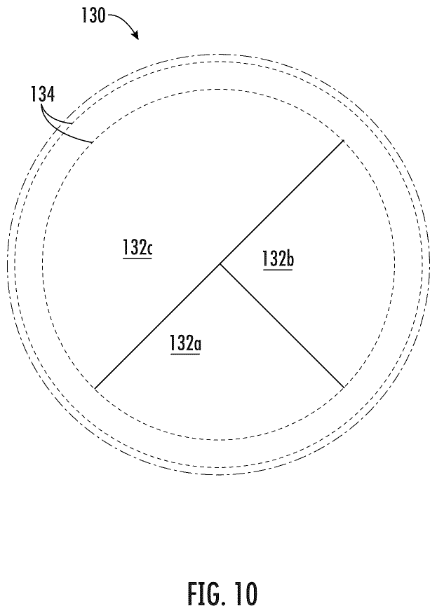

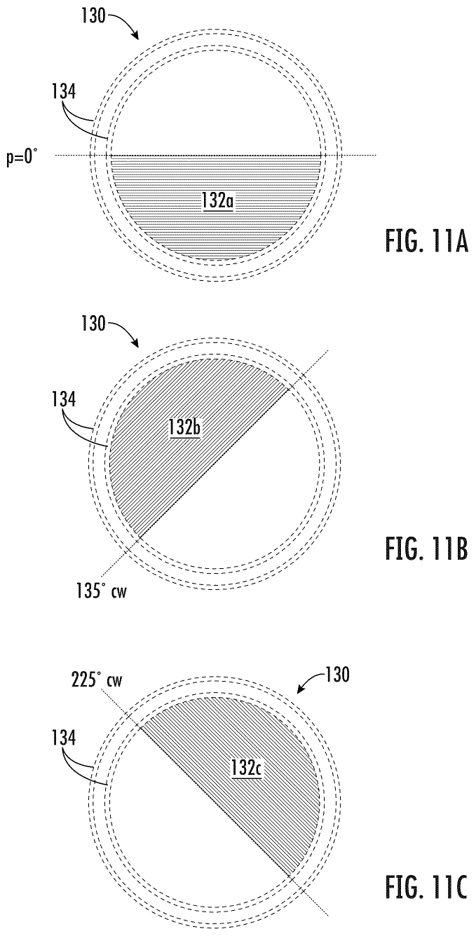

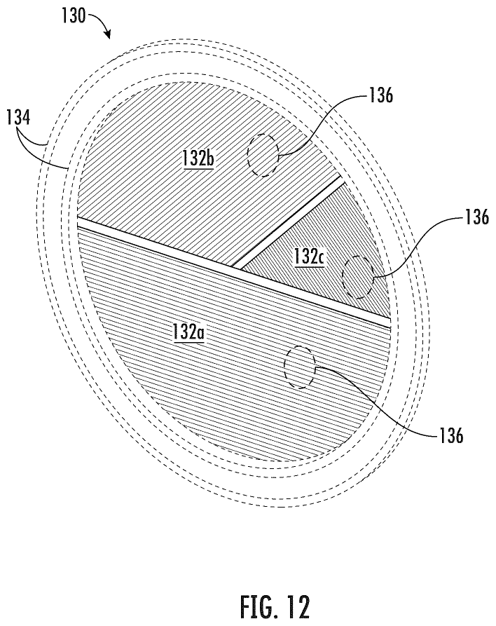

The arrangement and orientation of front opening flaps 132a, 132b, and 132c are important features that provide strength for securing and holding a milk collection bottle, such as milk collection bottles 200 shown in FIG. 1. For example, the arrangement and orientation of front opening flaps 132a, 132b, and 132c is such to provide as much flap overlap at the upper portion of front opening 130 as possible. However, another beneficial feature for providing strength may be the orientation of the grain of front opening flaps 132a, 132b, and 132c. For example , FIG. 12, FIG. 13, and FIG. 14 show another perspective view, front view, and back view, respectively, of front opening 130 and indicating a grain 136 of the three overlapping front opening flaps 132a, 132b, and 132c. In this example, the direction of grain 136 of each of the front opening flaps 132a, 132b, and 132c may be substantially parallel to the straight edge of each.

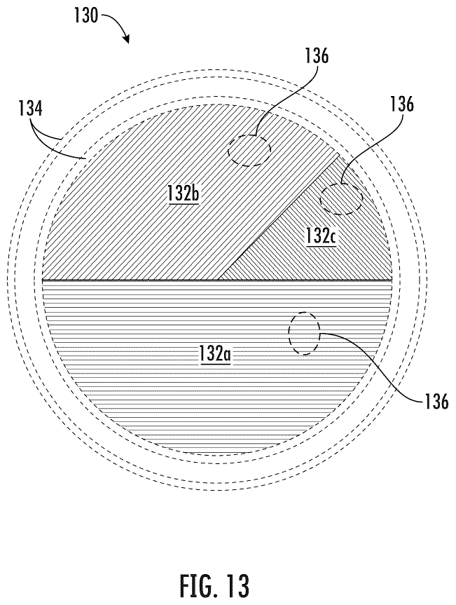

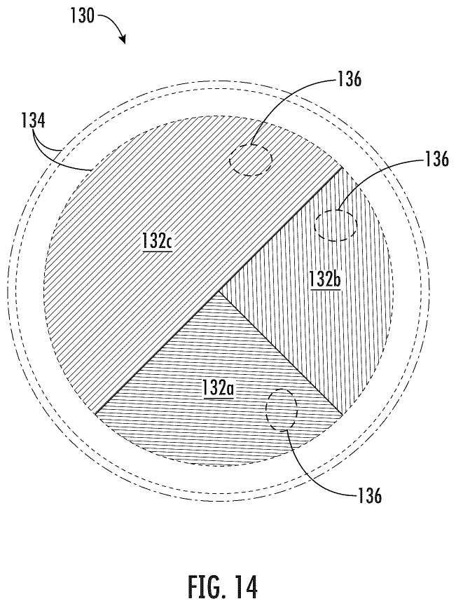

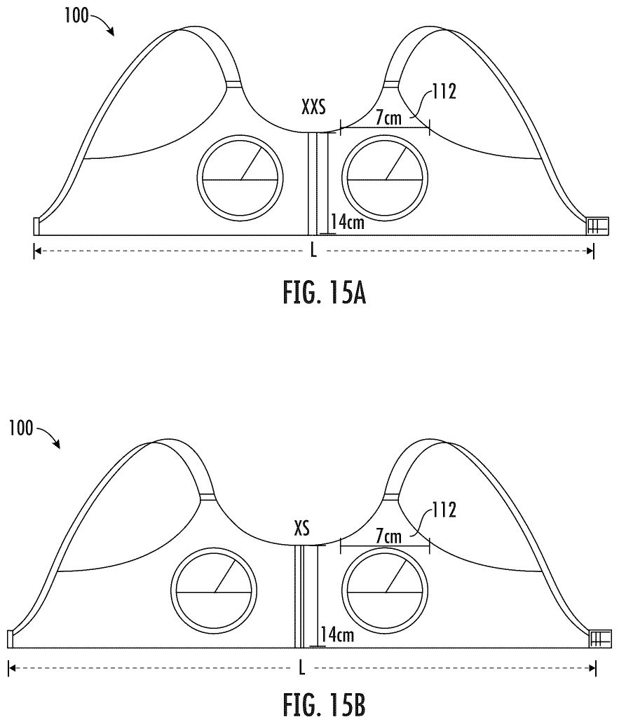

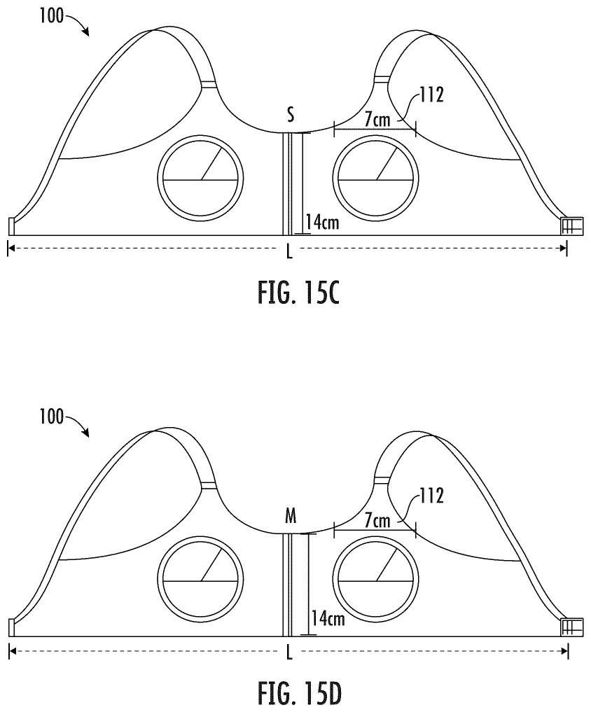

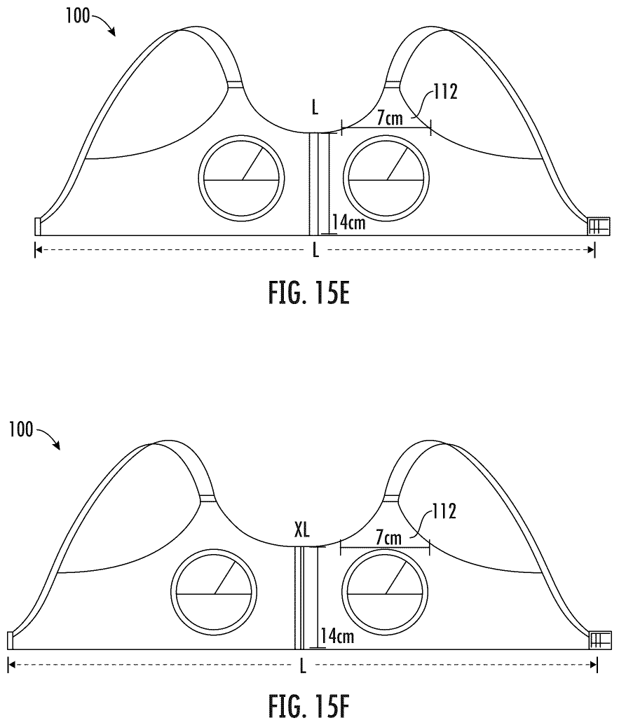

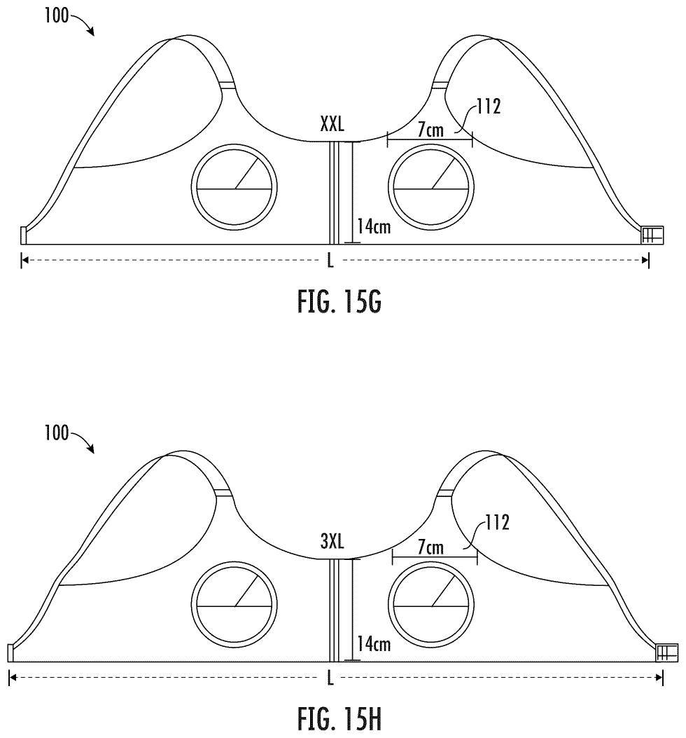

Referring now to FIG. 15A through FIG. 15H is front plan views showing examples of different sizes of hands-free pumping bra 100, in accordance with an embodiment of the invention. For example, in FIG. 15A through FIG. 15H, hands-free pumping bra 100 is shown in the unfastened from the back state and having an overall length L.

FIG. 15A shows an example of hands-free pumping bra 100 having an overall length L of, for example, about 54 cm for providing an extra-extra small (XXS) size.

FIG. 15B shows an example of hands-free pumping bra 100 having an overall length L of, for example, about 58 cm for providing an extra small (XS) size.

FIG. 15C shows an example of hands-free pumping bra 100 having an overall length L of, for example, about 62 cm for providing a small (S) size.

FIG. 15D shows an example of hands-free pumping bra 100 having an overall length L of, for example, about 66 cm for providing a medium (M) size.

FIG. 15E shows an example of hands-free pumping bra 100 having an overall length L of, for example, about 70 cm for providing a large (L) size.

FIG. 15F shows an example of hands-free pumping bra 100 having an overall length L of, for example, about 74 cm for providing an extra large (XL) size.

FIG. 15G shows an example of hands-free pumping bra 100 having an overall length L of, for example, about 78 cm for providing an extra-extra large (XXL) size.

FIG. 15H shows an example of hands-free pumping bra 100 having an overall length L of, for example, about 82 cm for providing an extra-extra-extra large (3XL) size.

In each example shown in FIG. 15A through FIG. 15H, the example overall length (L) maybe more or less than the above examples, and additional sizes may be included. Further, in each example shown in FIG. 15A through FIG. 15H, the height of the front center portion where right body panel 110R and left body panel 110L meet at front zipper 120 may be, for example, about 14 cm.

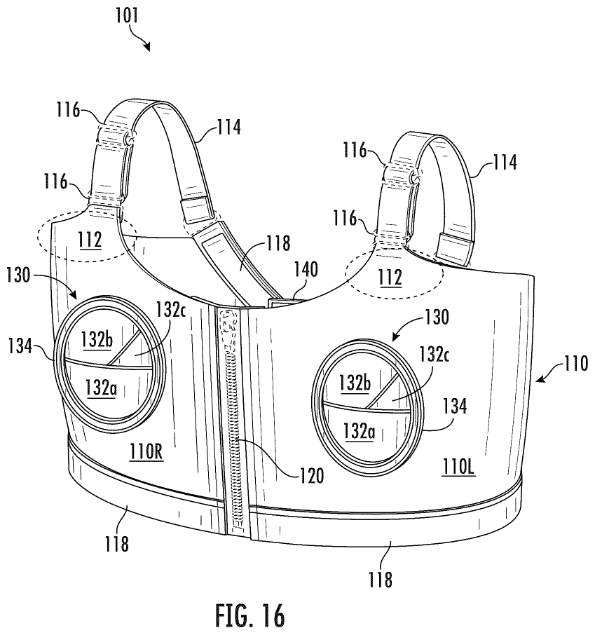

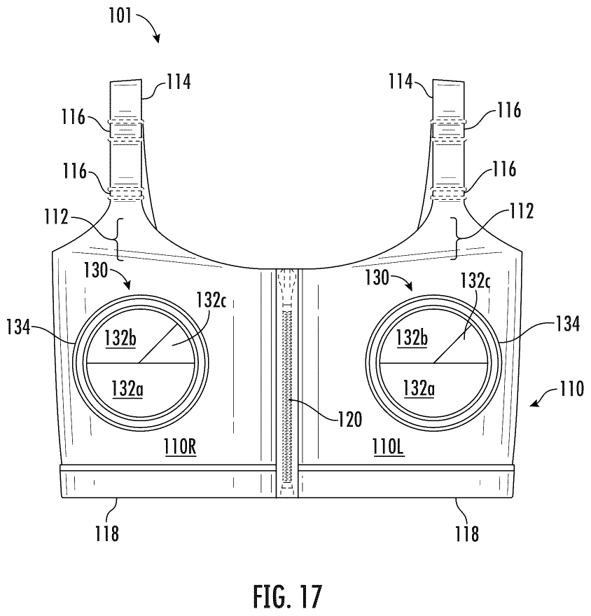

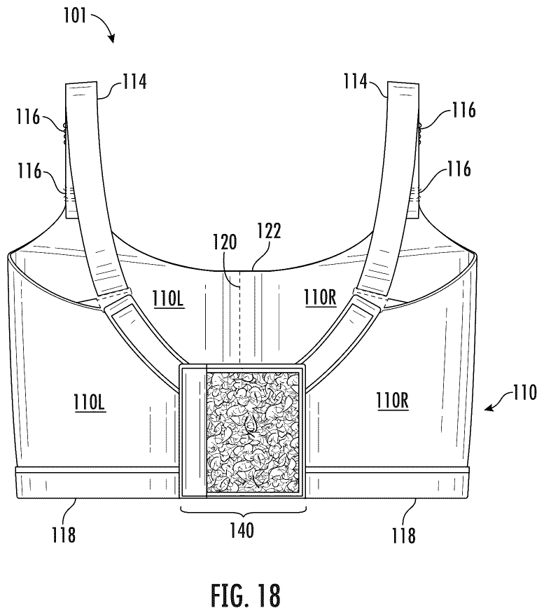

Hands-free pumping bra may be provided with different types of fastening in the back. In one example and referring now to FIG. 16 through FIG. 21, an example of a hands-free pumping bra 101 is provided that may include a hook and loop system, in accordance with an embodiment of the invention. In this example, hands-free pumping bra 101 may be substantially the same as hands-free pumping bra 100 shown and described above in FIG. 2 through FIG. 15H except that hook and eye system 124 is replaced with a hook and loop system 140. In one example, hook and loop system 140 may be VELCRO®.

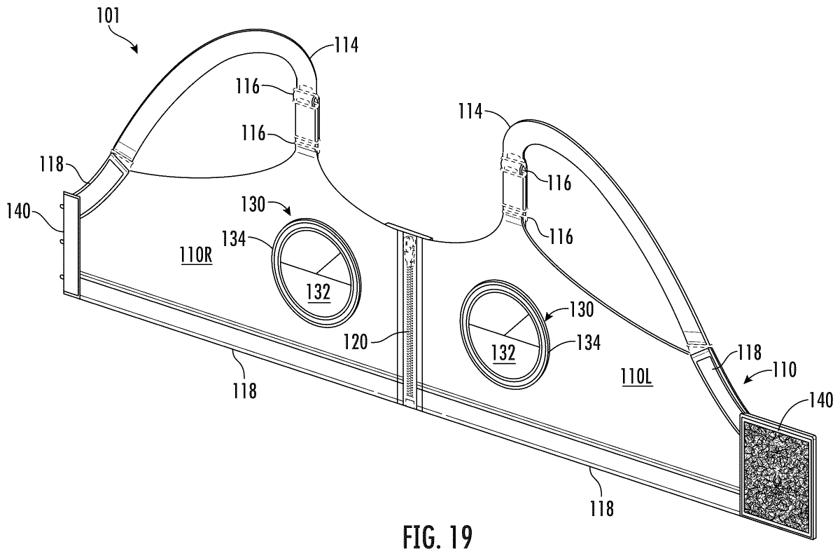

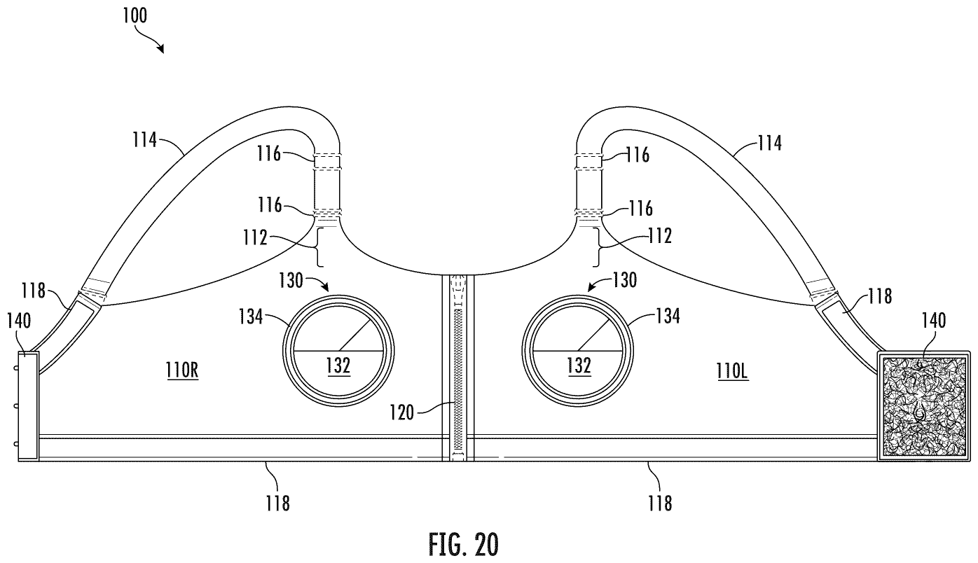

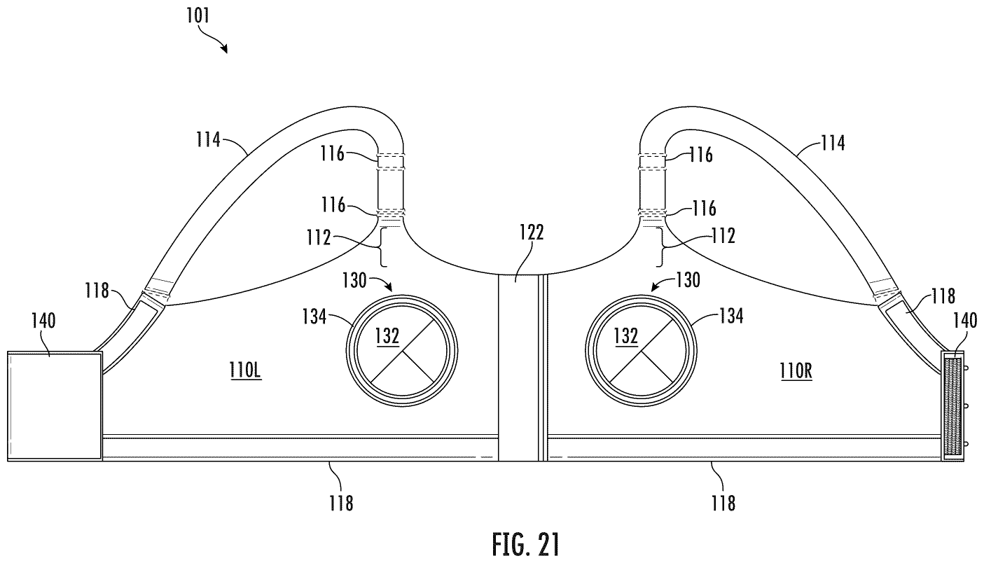

For example, FIG. 16, FIG. 17, and FIG. 18 is a perspective view, a front view, and a back view, respectively, of hands-free pumping bra 101 in a fastened state via an example of hook and loop system 140. Further, FIG. 19, FIG. 20, and FIG. 21 is a perspective view, a front plan view, and a back plan view, respectively, of hands-free pumping bra 101 in an unfastened from the back state.

In one example, a patch of hook and loop system 140 may be provided on one end of right body panel 110R and a complementary patch of hook and loop system 140 may be provided on one end of left body panel 110L, as shown in FIG. 19, FIG. 20, and FIG. 21 (or vice versa). Hook and loop system 140 provides adjustment for providing the best fit. Further, hands-free pumping bra 101 may be provided in the sizes shown in FIG. 15A through FIG. 15H.

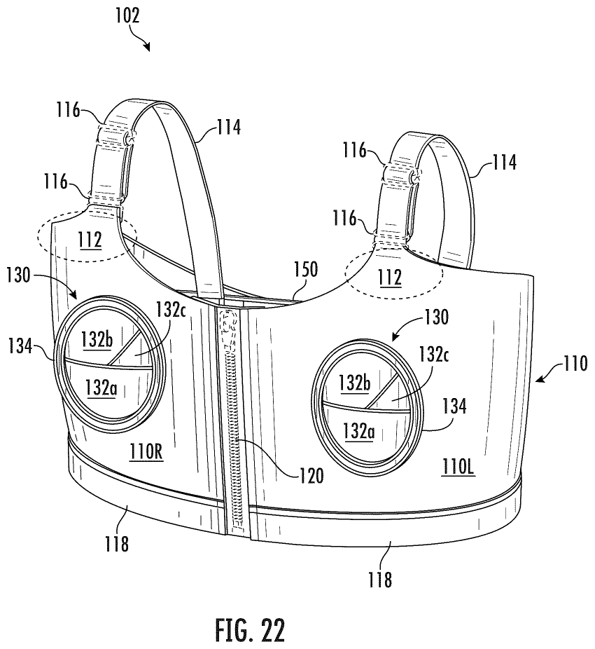

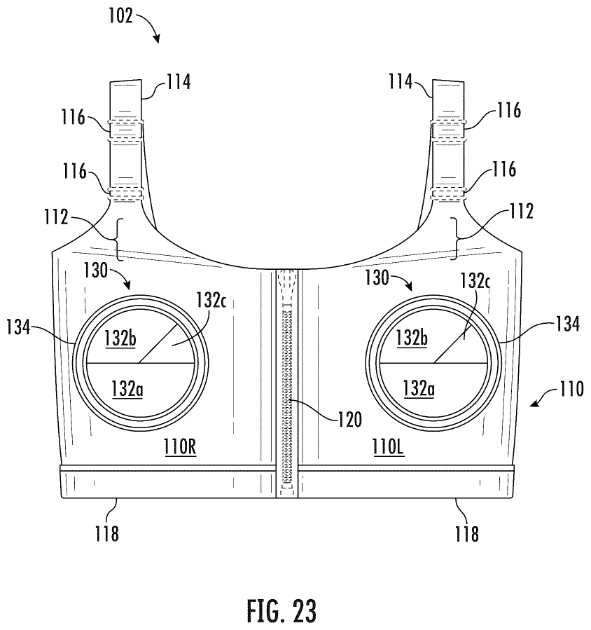

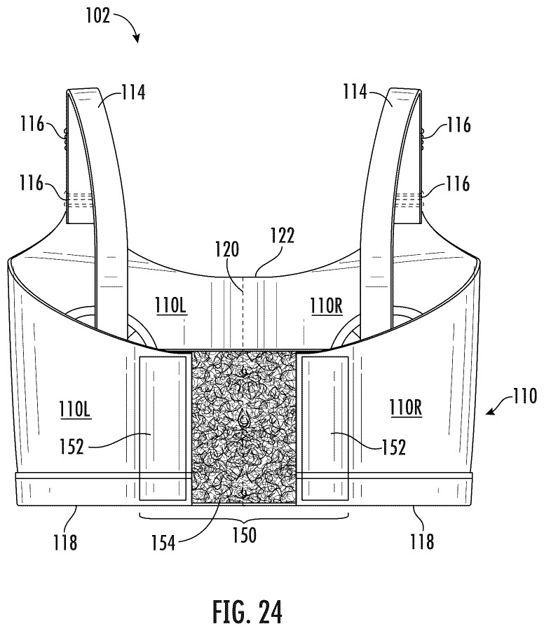

In another example and referring now to FIG. 22 through FIG. 27, an example of a hands-free pumping bra 102 is provided that may include a hook and loop system, in accordance with an embodiment of the invention. In this example, hands-free pumping bra 102 may include a hook and loop system 150 arranged as shown in FIG. 22 through FIG. 27. In one example, hook and loop system 150 may be VELCRO®.

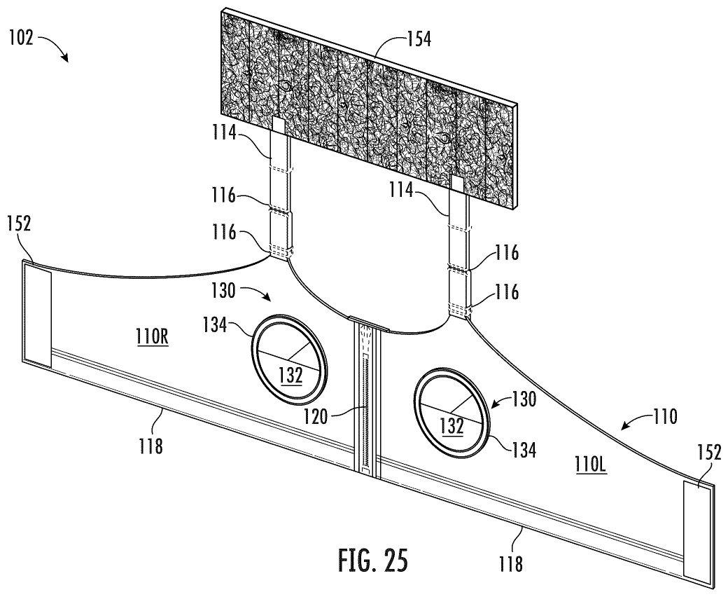

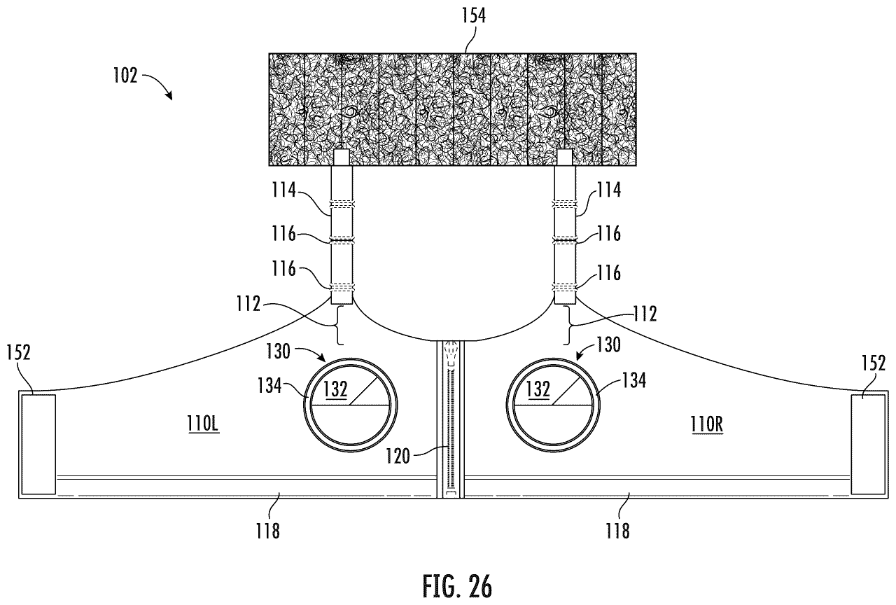

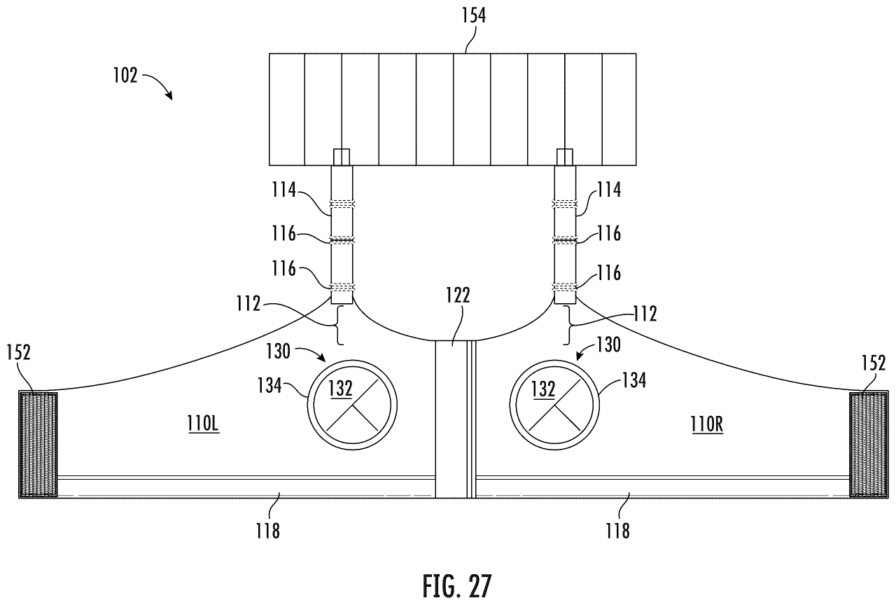

For example, FIG. 22, FIG. 23, and FIG. 24 is a perspective view, a front view, and a back view, respectively, of hands-free pumping bra 102 in a fastened state via hook and loop system 150. Further, FIG. 25, FIG. 26, and FIG. 27 is a perspective view, a front plan view, and a back plan view, respectively, of hands-free pumping bra 102 in an unfastened from the back state.

In this example, hook and loop system 150 provides a configuration that includes hook and loop portions 152 and 154. For example, and referring now to FIG. 25, FIG. 26, and FIG. 27, a hook and loop portion 152 may be provided on one end of right body panel 110R and another hook and loop portion 152 may be provided on one end of left body panel 110L. Further, in hands-free pumping bra 102, bra straps 114 may be configured differently than in hands-free pumping bras 100, 101. Here, one end of each of the two bra straps 114 may be attached to the top portion of right body panel 110R and left body panel 110L, respectively. Further, the other end of each of the two bra straps 114 may be attached to one hook and loop portion 154. In this example, the two hook and loop portions 152 may be small patches of VELCRO® while the one hook and loop portion 154 may be a large patch of VELCRO®.

FIG. 22, FIG. 23, and FIG. 24 show an example of the two hook and loop portions 152 attached to the one hook and loop portion 154. Accordingly, hook and loop system 150 may support hands-free pumping bra 102 being a one-size-fits-all pumping bra that may satisfy the sizes shown in FIG. 15A through FIG. 15H.

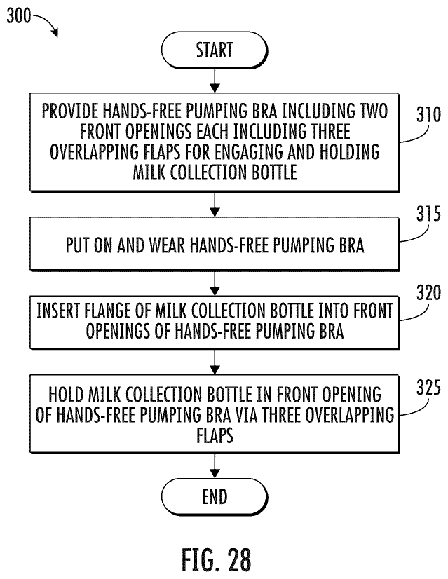

Referring now to FIG. 28 is a flow diagram of an example of a method 300 of using hands-free pumping bra 100, 101, and/or 102, in accordance with an embodiment of the invention. Method 300 may include, but is not limited to, the following steps.

At a step 310, a hands-free pumping bra is provided that includes two front openings with each front opening including three overlapping flaps for engaging and holding a milk collection bottle. In one example, and referring to FIG. 2 through FIG. 15H, hands-free pumping bra 100 may be provided that includes two front openings 130. Each front opening 130 including three overlapping front opening flaps 132 (e.g., 132a, 132b, 132c) for engaging and holding the flange of a milk collection bottle (e.g., flange 202 of milk collection bottle 200 shown in FIG. 1).

In another example, and referring to FIG. 16 through FIG. 21, hands-free pumping bra 101 may be provided that includes two front openings 130. Each front opening 130 including three overlapping front opening flaps 132 (e.g., 132a, 132b, 132c).

In yet another example, and referring to FIG. 22 through FIG. 27, hands-free pumping bra 102 may be provided that includes two front openings 130. Each front opening 130 including three overlapping front opening flaps 132 (e.g., 132a, 132b, 132c).

At a step 315, a woman puts on and wears the hands-free pumping bra. In one example, using front zipper 120 and hook and eye system 124 in the back, a nursing mother may put on and wear hands-free pumping bra 100 shown in FIG. 2 through FIG. 15H.

In another example, using front zipper 120 and hook and loop system 140 in the back, a nursing mother may put on and wear hands-free pumping bra 101 shown in FIG. 16 through FIG. 21.

In yet another example, using front zipper 120 and hook and loop system 150 in the back, a nursing mother may put on and wear hands-free pumping bra 102 shown in FIG. 22 through FIG. 27.

At a step 320, the woman inserts the flange of a milk collection bottle into the front opening of the hands-free pumping bra. In one example, and referring to FIG. 2 through FIG. 15H, a nursing mother inserts the flange of a milk collection bottle through the three overlapping front opening flaps 132 (e.g., 132a, 132b, 132c) of front opening 130 of hands-free pumping bra 100.

In another example, and referring to FIG. 16 through FIG. 21, a nursing mother inserts the flange of a milk collection bottle (see FIG. 1) through the three overlapping front opening flaps 132 (e.g., 132a, 132b, 132c) of front opening 130 of hands-free pumping bra 101.

In yet another example, and referring to FIG. 22 through FIG. 27, a nursing mother inserts the flange of a milk collection bottle through the three overlapping front opening flaps 132 (e.g., 132a, 132b, 132c) of front opening 130 of hands-free pumping bra 102.

At a step 325, the milk collection bottle is held into the front opening of the hands-free pumping bra via the three overlapping flaps.

In one example, and referring to FIG. 2 through FIG. 15H, the flange of a milk collection bottle (see FIG. 1) is held into front opening 130 of hands-free pumping bra 100 via the three overlapping front opening flaps 132 (e.g., 132a, 132b, 132c).

In another example, and referring to FIG. 16 through FIG. 21, the flange of a milk collection bottle (see FIG. 1) is held into front opening 130 of hands-free pumping bra 101 via the three overlapping front opening flaps 132 (e.g., 132a, 132b, 132c).

In yet another example, and referring to FIG. 22 through FIG. 27, the flange of a milk collection bottle (see FIG. 1) is held into front opening 130 of hands-free pumping bra 102 via the three overlapping front opening flaps 132 (e.g., 132a, 132b, 132c).

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

The terms “comprise,” “comprises,” “comprising,” “include,” “includes,” and “including,” are intended to be non-limiting, such that recitation of items in a list is not to the exclusion of other like items that may be substituted or added to the listed items.

Terms like “preferably,” “commonly,” and “typically” are not utilized herein to limit the scope of the claimed embodiments or to imply that certain features are critical or essential to the structure or function of the claimed embodiments. These terms are intended to highlight alternative or additional features that may or may not be utilized in a particular embodiment of the present invention.

The term “substantially” is utilized herein to represent the inherent degree of uncertainty that may be attributed to any quantitative comparison, value, measurement, or other representation and to represent the degree by which a quantitative representation may vary from a stated reference without resulting in a change in the basic function of the subject matter at issue.

Various modifications and variations of the disclosed methods, compositions and uses of the invention will be apparent to the skilled person without departing from the scope and spirit of the invention. Although the subject matter has been disclosed in connection with specific preferred aspects or embodiments, it should be understood that the subject matter as claimed should not be unduly limited to such specific aspects or embodiments.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A hands-free pumping bra garment, comprising:
a first body panel configured to be disposed over at least a portion of a first breast of a wearer, the first body panel comprising a first opening; and
a first covering coupled to the first body panel and disposed to cover the first opening, the first covering including a first flap attached to the first body panel and covering a first portion of the first opening, a second flap attached to the first body panel and covering a second portion of the first opening, and a third flap attached to the first body panel and covering a third portion of the first opening, wherein the first flap, the second flap, and the third flap at least partially overlap one another and collectively are required to cover the entire first opening, the first covering providing a first access opening configured to receive therethrough a portion of a first breast pump.

2. The garment of claim 1, wherein the first opening is substantially circular.

3. The garment of claim 1, wherein the first flap, the second flap, and the third flap are configured such that, when received therethrough, the received portion of the first breast pump is held in place without hand-held support.

4. The garment of claim 1, wherein the first opening is defined by an arrangement of reinforced stitching.

5. The garment of claim 1, wherein the first flap, the second flap, and the third flap each comprise a generally semicircular shape.

6. The garment of claim 1, wherein the first flap, the second flap, and the third flap are layered and orientated one to another such that all three together are required to cover the entire circular area of the first opening, wherein an absence of any one of the first flap, the second flap, or the third flap would leave a portion of the first opening uncovered.

7. The garment of claim 1, wherein a grain of a fabric of each of the first flap, the second flap, and the third flap is orientated to be substantially parallel to a straight edge of each of its respective one of the first flap, the second flap, and the third flap.

8. The garment of claim 1, further comprising:
a second body panel configured to be disposed over at least a portion of a second breast of a wearer, the second body panel comprising a second opening; and
a second covering coupled to the second body panel and disposed to cover the second opening, the second covering including a fourth flap attached to the second body panel and covering a first portion of the second opening, a fifth flap attached to the second body panel and covering a second portion of the second opening, and a sixth flap attached to the second body panel and covering a third portion of the second opening, wherein the fourth flap, the fifth flap, and the sixth flap at least partially overlap one another and collectively are required to cover the entire second opening, the second covering providing a second access opening configured to receive therethrough a portion of the first breast pump or a portion of a second breast pump.

9. The garment of claim 8, wherein the second opening is substantially circular.

10. The garment of claim 8, wherein the fourth flap, the fifth flap, and the sixth flap are configured such that when received therethrough the received portion of the first breast pump or the second breast pump is held in place without hand-held support.

11. The garment of claim 8, wherein the second opening is defined by an arrangement of reinforced stitching.

12. The garment of claim 8, wherein the fourth flap, the fifth flap, and the sixth flap each comprise a generally semicircular shape.

13. The garment of claim 8, wherein the fourth flap, the fifth flap, and the sixth flap are layered and orientated one to another such that all three together are required to cover the entire circular area of the second opening, wherein an absence of any one of the fourth flap, the fifth flap, or the sixth flap would leave a portion of the second opening uncovered.

14. The garment of claim 8, wherein a grain of a fabric of each of the fourth flap, the fifth flap, and the sixth flap is orientated to be substantially parallel to a straight edge of each of its respective one of the fourth flap, the fifth flap, and the sixth flap.

15. The garment of claim 8, further comprising a front fastener, wherein the front fastener is configured to fasten the first body panel and second body panel together at a front portion of the garment.

16. The garment of claim 15, wherein the front fastener comprises a zipper.

17. The garment of claim 8, further comprising a rear fastener, wherein the rear fastener is configured to fasten the first body panel and second body panel together in a back portion of the garment.

18. The garment of claim 17, wherein the rear fastener comprises one of or a combination of a hook and eye style fastener or a hook and loop style fastener.

19. A method of using a hands-free pumping bra garment, the method comprising:
providing a hands-free pumping bra garment, comprising:
a first body panel configured to be disposed over at least a portion of a first breast of a wearer, the first body panel comprising a first opening; and
a first covering coupled to the first body panel and disposed to cover the first opening, the first covering including a first flap attached to the first body panel and covering a first portion of the first opening, a second flap attached to the first body panel and covering a second portion of the first opening, and a third flap attached to the first body panel and covering a third portion of the first opening, wherein the first flap, the second flap, and the third flap at least partially overlap and collectively are required to cover the entire first opening, the first covering providing a first access opening configured to receive therethrough a portion of a first breast pump;
putting on the hands-free pumping bra garment by a user;
inserting a portion of the first breast pump into the first opening through the first flap, the second flap, and the third flap; and
supporting the inserted portion of the first breast pump in a handsfree manner via the first flap, second flap, and third flap. The method of claim 19, wherein the hands-free pumping bra garment further comprises:
a second body panel configured to be disposed over at least a portion of a second breast of a wearer, the second body panel comprising a second opening; and
a second covering coupled to the second body panel and disposed to cover the second opening, the second covering including a fourth flap attached to the second body panel and covering a first portion of the second opening, a fifth flap attached to the second body panel and covering a second portion of the second opening, and a sixth flap attached to the second body panel and covering a third portion of the second opening, wherein the fourth flap, the fifth flap, and the sixth flap at least partially overlap and collectively are required to cover the entire second opening, the second covering providing a second access opening configured to receive therethrough a portion of the first breast pump or a portion of a second breast pump.

21. The method of claim 20, further comprising: inserting a portion of the first breast pump or the second breast pump into the second opening through the fourth flap, the fifth flap, and the sixth flap; and holding the inserted portion of the first breast pump or the second breast pump in a handsfree manner via the fourth flap, the fifth flap, and the sixth flap.

22. A hands-free pumping bra, comprising:
a body portion configured to be disposed over at least a portion of a breast of a wearer, the body portion comprising an opening, wherein the opening is configured to be in substantial alignment with a nipple region of a wearer; and
a covering coupled to the body portion and disposed to cover the opening, the covering including three overlapping substantially semicircular-shaped flaps attached to the body portion, wherein the three overlapping semicircular-shaped flaps collectively are required to cover the entire opening, the covering providing an access opening configured to receive therethrough a portion of a breast pump.
